(19) United States
(12) Patent Application Publication
(10) Pub. No.: US 2008/0194470 A1
(43) Pub. Date: Aug. 14, 2008

(54) Nim811 In Cerebral Ischemia and Brain and Spinal Cord Injury

(75) Inventor: Peter Waldmeier, Ettingen (CH)

Correspondence Address: NOVARTIS;CORPORATE INTELLECTUAL PROPERTY, ONE HEALTH PLAZA 104/3, EAST HANOVER, NJ, 07936-1080, US

(73) Assignee: Novartis AG, East Hanover, NJ (US)

(21) Appl. No.: 10/599,676

(22) PCT Filed: Apr. 7, 2005
(86) PCT No.: PCT/EP2005/003664

§ 371 Date: Feb. 25, 2008

Related U.S. Application Data
(60) Provisional application No. 60/560,465, filed on Apr. 8, 2004.

(51) Int. Cl. A61K 38/08; C07K 7/64

(52) U.S. Cl. 514/11; 530/321

(57) ABSTRACT

Non-immunosuppressive, cyclophilin-binding cyclosporins, in particular, [Melle]⁴-Ciclosporin, are useful as neuroprotective agents, e.g., in the prevention or treatment of cerebral ischemia or traumatic brain or spinal cord injury.

The present invention relates to novel uses of cyclosporins and, in particular, to new pharmaceutical uses of non-immunosuppressive, cyclophilin binding cyclosporins.

Non-immunosuppressive, cyclophilin binding cyclosporins and their use in the treatment and prevention of AIDS and AIDS-related disorders are described in European Patent No. 0484281 B (EP'281), which includes a general description of the cyclosporin class of compounds, their nomenclature and mode of action. The disclosure of EP'281, in particular, the general description referred to above and other parts of the description referred to hereinafter, is included by reference in the teaching of the present application.

Surprisingly, it has now been found that cyclosporins which bind to cyclophilin, but are not immunosuppressive, are useful as neuroprotective agents, e.g., in ischemic brain damage, traumatic brain and spinal cord injury and stroke.

A cyclosporin is considered as binding to cyclophilin if it binds to human recombinant cyclophilin at least one-fifth, as well as does Ciclosporin (also referred to as cyclosporin A) in the competitive ELISA test described by Quesniaux, Eur J Immunol, Vol. 17, pp. 1359-1365 (1987). In this test, the cyclosporin to be tested is added during the incubation of cyclophilin with coated BSA-Ciclosporin and the concentration required to give a 50% inhibition of the control reaction without competitor is calculated (IC₅₀). The results are expressed as the Binding Ratio (BR), which is the log to the base 10 (log₁₀) of the ratio of the IC₅₀ of the test compound and the IC₅₀ in a like test using Ciclosporin in place of the test cyclosporin. Thus a BR of 1.0 indicates that the test compound binds cyclophilin one factor of ten less well than does Ciclosporin, and a negative value indicates binding stronger than that of Ciclosporin.

The cyclosporins active as neuroprotective agents have a BR lower than 0.7, (since log₁₀ 5=0.7 approx.), preferably equal to or lower than zero.

A cyclosporin is considered to be non-immunosuppressive when it has an activity in the mixed lymphocyte reaction (MLR) of no more than 5%, preferably no more than 2%, that of Ciclosporin. The MLR is described by T. Meo, Immunological Methods, Lefkovits and Peris, Eds., Academic Press, NY, pp. 227-239 (1979). Spleen cells (0.5×10⁶) from Balb/c mice (female, 8-10 weeks) are co-incubated for 5 days with 0.5×10⁶ irradiated (2000 rads) or mitomycin C-treated spleen cells from CBA mice (female, 8-10 weeks). The irradiated allogeneic cells induce a proliferative response in the Balb/c spleen cells which can be measured by labeled precursor incorporation into the DNA. Since the stimulator cells are irradiated (or mitomycin C-treated) they do not respond to the Balb/c cells with proliferation but do retain their antigenicity. The IC₅₀ found for the test compound in the MLR is compared with that found for Ciclosporin in a parallel experiment.

It has been found that compounds which are judged as non-immunosuppressive in the MLR above are often inactive in an IL-2 reporter gene assay, and thus an IL-2 reporter gene assay may be used, e.g., as a primary screen, for selection of non-immunosuppressive, cyclophilin-binding cyclosporin compounds for use in the invention.

The non-immunosuppressive, cyclophilin-binding cyclosporin compounds which are active as neuroprotective agents, e.g., as inhibitors of neuronal cell death during ischemia or traumatic brain or spinal cord injury or as a result of a stroke are hereinafter referred to as “active compounds”.

The active compounds are therefore useful in the treatment of any clinical condition involving a component of cerebral anoxia, hypoxia and/or ischemia, e.g., ischemic damage to grey and white matter, stroke, reperfusion injury, subarachnoid hemorrhage, brain and spinal cord injury/trauma, high intracranial pressure, multi-infarct dementia or vascular dementia and any surgical procedure potentially associated with cerebral anoxia, hypoxia and/or ischemia, e.g., cardiac bypass, operations on extracerebral vessels.

It is found that many of the active compounds have structures differing from that of Ciclosporin specifically at the 4 and/or 5 positions. Other positions at which the structures of the active compounds may differ from that of Ciclosporin are positions 6 and 7.

One group of active compounds are cyclosporins in which the MeLeu group at position 4 is replaced by a different N-methylated amino acid, e.g., γ-hydroxy-MeLeu, Melle, MeVal, MeThr, MeAla, MeTyr or MeTyr(O—PO(OH)₂) or Pro. In addition to Melle and MeThr, the allo-forms Mealle and MeaThr may also be used. In the allo-form, the stereochemistry at the β-position has the opposite configuration to that of the natural amino acid, so that the normal form and the allo-form constitute a pair of diastereoisomers.

A further group of active compounds is that in which Val at the 5-position is replaced by an N-alkyl-, preferably N-methyl-, amino acid. Preferably, the amino acid which is N-alkylated is Val or Leu. Preferably the hydrogen of the imino group of [Val]⁵ is replaced by a non-branched C₁₋₆alkyl group, preferably methyl, ethyl or n-propyl, particularly methyl. The latter preferred group of active compounds are all novel.

Additionally or alternatively, certain active compounds may differ from Ciclosporin at the 1, 2, 3 and/or 6 positions.

A particular class of active compounds for use in the present invention are Ciclosporin derivatives of formula (A):

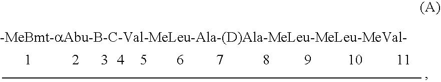

wherein B is an amino acid residue of formula (B):

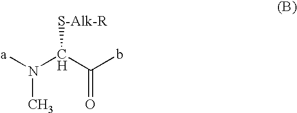

wherein
a denotes the bond to the αAbu residue in position 2;
b denotes the bond to the residue C in the 4 position;
Alk represents straight- or branched-chain alkylene containing from 2-6 carbon atoms or cycloalkylene containing from 3-6 carbon atoms; and
R represents
   a carboxy or alkyloxycarbonyl radical;
   a radical —NR₁R₂,
   in which
      R₁ and R₂ are the same or different and represent hydrogen, alkyl, C₂₋₄alkenyl, C₃₋₆cycloalkyl, phenyl (optionally substituted by halogen, alkoxy, alkoxycarbonyl, amino, alkylamino or dialkylamino) or a benzyl or saturated or unsaturated heterocyclyl radical containing 5- or 6-ring atoms and 1-3 heteroatoms, or
      R₁ and R₂ form, together with the nitrogen atom to which they are attached, a saturated or unsaturated heterocycle containing 4-6 ring atoms and optionally containing a further heteroatom selected from nitrogen, oxygen or sulphur and optionally substituted by alkyl, phenyl or benzyl;
   a radical of formula:

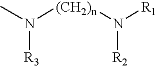

wherein
   R₁ and R₂ are as defined above;
   R₃ represents hydrogen or an alkyl radical; and
   n is a whole number from 2-4; and
alkyl denotes straight- or branched-chain alkyl containing from 1-4 carbon atoms;
C is MeLeu or 4-hydroxy-MeLeu;
   and the pharmaceutically acceptable salts thereof.

This class of Ciclosporin derivatives is further described in published International Patent Applications Nos. WO 98/28328, WO 98/28329 and WO 98/28330. A particularly preferred compound of this class is the compound of formula (A),

in which

B is the amino acid residue of formula (B′):

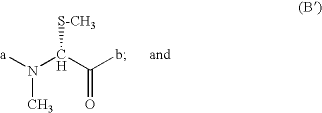

and

C is the amino acid residue 4-hydroxy-MeLeu.

A particularly preferred group of active compounds is constituted by the compounds of formula (I):

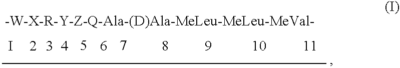

in which
W is MeBmt, dihydro-MeBmt or 8′-hydroxy-MeBmt;
X is αAbu, Val, Thr, Nva or O-methyl threonine (MeOThr);
R is Sar or (D)-MeAla;
Y is MeLeu, γ-hydroxy-MeLeu, Melle, MeVal, MeThr, MeAla, Me Tyr, MeTyr(O—PO(OH)₂), Mealle or MeaThr or Pro;
Z is Val, Leu, N-Alk-Val or N-Alk-Leu, wherein Alk represents Me or Me substituted by vinyl optionally substituted by phenyl, or an N, S or O heteroaryl containing 6 ring members, or phenyl optionally substituted by halogen; and
Q is MeLeu, γ-hydroxy-MeLeu or MeAla;
   and the pharmaceutically acceptable salts thereof.

The groups W, X, Y, Z and Q have, independently, the following preferred significances:

W is preferably W′, where W′ is MeBmt or dihydro-MeBmt;
X is preferably X′, where X′ is αAbu or Nva, more preferably X″, where X″ is αAbu;
Y is preferably Y′, where Y′ is γ-hydroxy-MeLeu, MeVal, MeThr, MeAla or MeTyr(O—PO(OH)₂);
Z is preferably Z′, where Z′ is Val or MeVal; and
Q is preferably Q′, where Q′ is MeLeu;

One especially preferred group of active compounds are the compounds of formula (I), in which

W is W′;

X is X′;

Y is Y′;

Z is Z′; and

Q is Q′.

Particularly preferred active compounds of formula (I) are:

a) [dihydro-MeBmt]¹-[γ-hydroxy-MeLeu]⁴-Ciclosporin;

### b) [MeVal]⁴-Ciclosporin;

### c) [Melle]⁴-Ciclosporin;

### d) [MeThr]⁴-Ciclosporin;

e) [γ-hydroxy-MeLeu]⁴-Ciclosporin;
f) [Nva]²-[γ-hydroxy-MeLeu]⁴-Ciclosporin;
g) [γ-hydroxy-MeLeu]⁴-[γ-hydroxy-MeLeu]⁶-Ciclosporin;

### h) [MeVal]⁵-Ciclosporin;

### i) [MeOThr]²-[(D)MeAla]³-[MeVal]⁵-Ciclosporin;

j) [8′-hydroxy-MeBmt]¹-Ciclosporin;

### k) [MeAla]⁶-Ciclosporin;

### l) [DMeAla]³-[MeTyr(OPO(OH)₂)]⁴-Ciclosporin;

### m) [N-Benzyl-Val]⁵-Ciclosporin;

### n) [N-5-Fluoro-Benzyl-Val]⁵-Ciclosporin;

### o) [N-Allyl-Val]⁵-Ciclosporin;

### p) [N-3-Phenyl-Allyl-Val]⁵-Ciclosporin; or

### q) [Pro]⁴-Ciclosporin.

Especially preferred active compounds are [Melle]⁴-Ciclosporin and [γ-hydroxy-MeLeu]⁴-Ciclosporin, most especially [Melle]⁴-Ciclosporin.

In addition to the compounds of formula (I), preferred active compounds include, e.g.,

r) [γ-hydroxy-MeLeu]⁹-Ciclosporin.

The active compounds may be obtained by methods including:

### 1) Fermentation;

### 2) Biotransformation;

### 3) Derivatisation;

### 4) Partial Synthesis; and

### 5) Total Synthesis.

These methods are described generally and more specifically in Examples 1-10 of EP'281. This general description and the teaching of these Examples are incorporated by reference in the present application. Example 11 of EP'281 describes measurement of the immunosuppressive and cyclophilin-binding activities of representative active compounds relative to Ciclosporin, and the teaching of this examples is also included within the disclosure of the present application.

Thus the invention provides use of a non-immunosuppressive, cyclophilin-binding cyclosporin in the manufacture of a medicament for treating or preventing ischemic brain damage, traumatic brain or spinal cord injury or stroke.

The invention further provides a method for the treatment or the prevention of ischemic brain damage, traumatic brain or spinal cord injury or stroke in a patient suffering or at risk of such a disease or condition, comprising administering to said patient an effective amount of an active compound of the invention.

The active compound may be administered by any conventional route, in particular, enterally, e.g., orally, e.g., in the form of solutions for drinking, tablets or capsules; or parenterally, e.g., in the form of injectable solutions or suspensions. By the intravenous route an indicated daily dosage may be from 1-20 mg/kg, preferably from 3-10 mg/kg, and by the oral route from 1-50 mg/kg, preferably from 10-30 mg/kg.

The toxicity of the active compounds is believed to be less to that of Ciclosporin. As the active compounds are not immunosuppressive, certain side effects of Ciclosporin related to immunosuppression are avoided. Other side effects associated with Ciclosporin, particularly nephrotoxicity and central nervous system toxicity in long term use, are conveniently less than with Ciclosporin.

Preferred galenic formulations for the active compounds include those based on microemulsions as described in British Patent Application No. 2 222 770A (GB'770), which include topical, as well as oral forms; also oral and injectable forms obtained from solid solutions comprising a fatty acid saccharide monoester, e.g., saccharose monolaurate, as described in British Patent Application No. 2 209 671A. Suitable unit dosage forms for oral administration comprise, e.g., from 25-200 mg active compound per dosage.

Formulation Examples A, B, C and D of EP'281 are incorporated herein by reference:

The individual components of these formulations, as well as the methods for their preparation, are fully described in GB'770, the contents of which are incorporated herein by reference.

The usefulness of the active compounds as neuroprotective agents may be demonstrated in in vivo or in vitro tests, e.g.,
1. Spinal cord injury model (in vivo)
   Adult Lewis rats are injured microsurgically by transecting the dorsal half of the spinal cord bilaterally at the level of the eighth thoracic vertebra. Laminectomy, anesthesia and surgery are described in Schnell and Schwab, Eur J Neurosci, Vol. 5, pp. 1156-1171 (1993). The active compounds may be tested for their ability to reduce neuronal cell death in this model.
2. Middle cerebral artery (MCA) occlusion model (in vivo)
   The active compounds are tested for their ability to reduce ischemia-induced neuronal damage and ensuing symptoms in the MCA occlusion model in rats, e.g., at a dosage of 1-30 mg/kg i.p., i.v. and p.o. [cf. Tamura et al., J Cereb Blood Flow Metabol, Vol. 1, pp. 53-60 (1981); and Sauter and Rudin, Stroke, Vol. 17, pp. 1228-1234 (1986)].
3. Inhibition of mitochondrial permeability transition in isolated rodent brain-derived mitochondria and prevention of cell death in an in vitro model of ischemic brain damage, e.g. as described in Rytter et al., JCBF, Vol. 23, pp. 23-33. In this model, active compounds, e.g., [Melle]⁴-Ciclosporin and [γ-hydroxy-MeLeu]⁴-Ciclosporin, are inhibitors of calcium-induced mitochondrial swelling under energized and de-energized conditions, comparable to the effects of cyclosporine A. Active compounds ameliorate selective CA1 cell death in organotypic mouse hippocampal slices exposed to 12 minutes of oxygen and glucose deprivation.

The active compounds of the invention can be provided alone, or in combination, or in sequential combination with other agents. For example, the active compounds of the invention can be administered in combination with anti-inflammatory agents, such as but not limited to, corticosteroids following stroke or spinal cord injury as a means for blocking further neuronal damage and inhibition of axonal regeneration; neurotrophic factors, such as NGF; BDNF or other drugs for neurodegenerative diseases, such as Exelon™ or Levodopa. As used herein, two agents are said to be administered in combination when the two agents are administered simultaneously or are administered independently in a fashion such that the agents will act at the same time.

The structure of the active ingredients identified by code nos., generic or trade names may be taken from the actual edition of the standard compendium “The Merck Index” or from databases, e.g., Patents International, e.g., IMS World Publications. The corresponding content thereof is hereby incorporated by reference. Any person skilled in the art is fully enabled to identify the active ingredients and, based on these references, likewise enabled to manufacture and test the pharmaceutical indications and properties in standard test models, both in vitro and in vivo.

For the indications mentioned above, the appropriate dosage will, of course, vary depending upon, e.g., the particular molecule of the invention to be employed, the mode of administration and the nature and severity of the condition being treated.

1. Use of a non-immunosuppressive, cyclophilin-binding cyclosporin in the manufacture of a medicament for treating or preventing ischemic brain damage, traumatic brain or spinal cord injury or stroke.

2. A method for the treatment or the prevention of ischemic brain damage or traumatic brain or spinal cord injury in a patient suffering from or at risk of suffering from such a disease or condition, comprising administering to said patient an effective amount of a non-immunosuppressive, cyclophilin-binding cyclosporin.

3. A use according to claim 1, in which the non-immunosuppressive, cyclophilin-binding cyclosporin is a compound of formula (A): 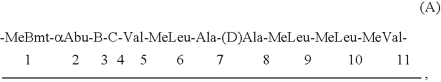
wherein B is an amino acid residue of formula (B): 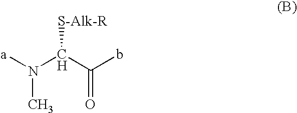
wherein
a denotes the bond to the αAbu residue in position 2;
b denotes the bond to the residue C in the 4 position;
Alk represents straight- or branched-chain alkylene containing from 2-6 carbon atoms or cycloalkylene containing from 3-6 carbon atoms; and
R represents
a carboxy or alkyloxycarbonyl radical;
a radical —NR₁R₂,
in which
R₁ and R₂ are the same or different and represent hydrogen, alkyl, C₂₋₄alkenyl, C₃₋₆cycloalkyl, phenyl (optionally substituted by halogen, alkoxy, alkoxycarbonyl, amino, alkylamino or dialkylamino) or a benzyl or saturated or unsaturated heterocyclyl radical containing 5- or 6-ring atoms and 1-3 heteroatoms, or
R₁ and R₂ form, together with the nitrogen atom to which they are attached, a saturated or unsaturated heterocycle containing 4-6 ring atoms and optionally containing a further heteroatom selected from nitrogen, oxygen or sulphur and optionally substituted by alkyl, phenyl or benzyl;
a radical of formula: 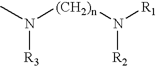
wherein
R₁ and R₂ are as defined above;
R₃ represents hydrogen or an alkyl radical; and
n is a whole number from 2-4; and
alkyl denotes straight- or branched-chain alkyl containing from 1-4 carbon atoms;
C is MeLeu or 4-hydroxy-MeLeu;
and the pharmaceutically acceptable salts thereof.

4. A use according to claim 1, in which the non immunosuppressive, cyclophilin-binding cyclosporin is a compound of formula (I): 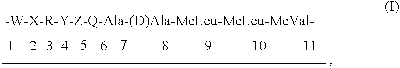
in which
W is MeBmt, dihydro-MeBmt or 8′-hydroxy-MeBmt;
X is αAbu, Val, Thr, Nva or O-methyl threonine (MeOThr);
R is Sar or (D)-MeAla;
Y is MeLeu, γ-hydroxy-MeLeu, Melle, MeVal, MeThr, MeAla, Me Tyr, MeTyr(O—PO(OH)₂), Mealle or MeaThr or Pro;
Z is Val, Leu, N-Alk-Val or N-Alk-Leu, wherein Alk represents Me or Me substituted by vinyl optionally substituted by phenyl, or an N, S or O heteroaryl containing 6 ring members, or phenyl optionally substituted by halogen; and
Q is MeLeu, γ-hydroxy-MeLeu or MeAla.

5. A use according to claim 1, in which the non-immunosuppressive, cyclophilin-binding cyclosporin is a compound selected from the group comprising:
a) [dihydro-MeBmt]-[γ-hydroxy-MeLeu]⁴-Ciclosporin;
b) [MeVal]⁴-Ciclosporin;
c) [Melle]⁴-Ciclosporin;
d) [MeThr]⁴-Ciclosporin;
e) [γ-hydroxy-MeLeu]⁴-Ciclosporin;
f) [Nva]²-[γ-hydroxy-MeLeu]⁴-Ciclosporin;
g) [γ-hydroxy-MeLeu]⁴-[γ-hydroxy-MeLeu]⁶-Ciclosporin;
h) [MeVal]⁵-Ciclosporin;
i) [MeOThr]²-[(D)MeAla]³-[MeVal]⁵-Ciclosporin;
j) [8′-hydroxy-MeBmt]¹-Ciclosporin;
k) [MeAla]⁶-Ciclosporin;
l) [DMeAla]³-[MeTyr(OPO(OH)₂)]⁴-Ciclosporin;
m) [N-Benzyl-Val]⁵-Ciclosporin;
n) [N-5-Fluoro-Benzyl-Val]⁵-Ciclosporin;
o) [N-Allyl-Val]⁵-Ciclosporin;
p) [N-3-Phenyl-Allyl-Val]⁵-Ciclosporin;
q) [Pro]⁴-Ciclosporin or
r) [γ-hydroxy-MeLeu]⁹-Ciclosporin.

6. A use according to claim 1, in which the non-immunosuppressive, cyclophilin-binding cyclosporin is [Melle]⁴-Ciclosporin or [γ-hydroxy-MeLeu]⁴-Ciclosporin.

7. A use according to claim 2, in which the non-immunosuppressive, cyclophilin-binding cyclosporin is a compound of formula (A): 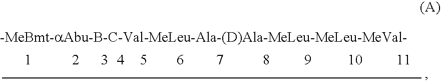
wherein B is an amino acid residue of formula (B): 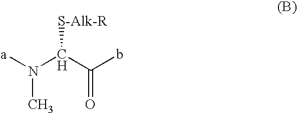
wherein
a denotes the bond to the αAbu residue in position 2;
b denotes the bond to the residue C in the 4 position;
Alk represents straight- or branched-chain alkylene containing from 2-6 carbon atoms or cycloalkylene containing from 3-6 carbon atoms; and
R represents
a carboxy or alkyloxycarbonyl radical;
a radical —NR₁R₂,
in which
R₁ and R₂ are the same or different and represent hydrogen, alkyl, C₂₋₄alkenyl, C₃₋₆cycloalkyl, phenyl (optionally substituted by halogen, alkoxy, alkoxycarbonyl, amino, alkylamino or dialkylamino) or a benzyl or saturated or unsaturated heterocyclyl radical containing 5- or 6-ring atoms and 1-3 heteroatoms, or
R₁ and R₂ form, together with the nitrogen atom to which they are attached, a saturated or unsaturated heterocycle containing 4-6 ring atoms and optionally containing a further heteroatom selected from nitrogen, oxygen or sulphur and optionally substituted by alkyl, phenyl or benzyl;
a radical of formula: 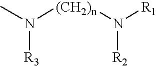
wherein
R₁ and R₂ are as defined above;
R₃ represents hydrogen or an alkyl radical; and
n is a whole number from 2-4; and
alkyl denotes straight- or branched-chain alkyl containing from 1-4 carbon atoms;
C is MeLeu or 4-hydroxy-MeLeu;
and the pharmaceutically acceptable salts thereof.
A use according to claim 1, in which the non immunosuppressive, cyclophilin-binding cyclosporin is a compound of formula (I): 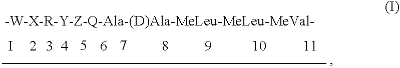
in which
W is MeBmt, dihydro-MeBmt or 8′-hydroxy-MeBmt;
X is αAbu, Val, Thr, Nva or O-methyl threonine (MeOThr);
R is Sar or (D)-MeAla;
Y is MeLeu, γ-hydroxy-MeLeu, Melle, MeVal, MeThr, MeAla, Me Tyr, MeTyr(O—PO(OH)₂), Mealle or MeaThr or Pro;
Z is Val, Leu, N-Alk-Val or N-Alk-Leu, wherein Alk represents Me or Me substituted by vinyl optionally substituted by phenyl, or an N, S or O heteroaryl containing 6 ring members, or phenyl optionally substituted by halogen; and
Q is MeLeu, γ-hydroxy-MeLeu or MeAla.

8. A use according to claim 2, in which the non-immunosuppressive, cyclophilin-binding cyclosporin is a compound selected from the group comprising:
a) [dihydro-MeBmt]-[γ-hydroxy-MeLeu]⁴-Ciclosporin;
b) [MeVal]⁴-Ciclosporin;
c) [Melle]⁴-Ciclosporin;
d) [MeThr]⁴-Ciclosporin;
e) [γ-hydroxy-MeLeu]⁴-Ciclosporin;
f) [Nva]²-[γ-hydroxy-MeLeu]⁴-Ciclosporin;
g) [γ-hydroxy-MeLeu]⁴-[γ-hydroxy-MeLeu]⁶-Ciclosporin;
h) [MeVal]⁵-Ciclosporin;
i) [MeOThr]²-[(D)MeAla]³-[MeVal]⁵-Ciclosporin;
j) [8′-hydroxy-MeBmt]¹-Ciclosporin;
k) [MeAla]⁶-Ciclosporin;
l) [DMeAla]³-[MeTyr(OPO(OH)₂)]⁴-Ciclosporin;
m) [N-Benzyl-Val]⁵-Ciclosporin;
n) [N-5-Fluoro-Benzyl-Val]⁵-Ciclosporin;
o) [N-Allyl-Val]⁵Ciclosporin;
p) [N-3-Phenyl-Allyl-Val]⁵-Ciclosporin;
q) [Pro]⁴-Ciclosporin or
r) [γ-hydroxy-MeLeu]⁹-Ciclosporin.

9. A use according to claim 2, in which the non-immunosuppressive, cyclophilin-binding cyclosporin is [Melle]⁴-Ciclosporin or [γ-hydroxy-MeLeu]⁴-Ciclosporin.
